(19) United States
(12) Patent Application Publication
(10) Pub. No.: US 2023/0272024 A1
(43) Pub. Date: Aug. 31, 2023

(54) FIBROBLAST GROWTH FACTOR 7 PEPTIDE

(71) Applicants: University of Florida Research Foundation, Incorporated, Gainesville, FL (US); University of Pittsburgh - Of the Commonwealth System of Higher Education, Pittsburgh, PA (US)

(72) Inventors: Lori P. RICE, Gainesville, FL (US); David A. OSTROV, Gainesville, FL (US); Carlton BATES, Wexford, PA (US)

(21) Appl. No.: 18/005,499

(22) PCT Filed: Jul. 9, 2021
(86) PCT No.: PCT/US2021/041070

§ 371 Date: Jan. 13, 2023

Related U.S. Application Data
(60) Provisional application No. 63/052,103, filed on Jul. 15, 2020.

(51) Int. Cl. C07K 14/50; A61K 47/20; A61P 43/00

(52) U.S. Cl. CPC C07K 14/50; A61K 47/20; A61P 43/00

(57) ABSTRACT

Described are peptide agonists for fibroblast growth factor receptor 2 (FGFR2) 111b isoform (FGFR2111b), compositions containing the peptide FGFR2111b agonists and methods of using the FGFR2111b agonists and compositions to treat or prevent epithelial damage. Specifically, the peptide agonist comprising an fibroblast growth factor 7 (FGF7) peptide. Further disclosed are compositions comprising the FGF7 peptides and methods of using the compositions for treating a subject having epithelial damage.

### CROSS-REFERENCE TO RELATED APPLICATIONS

This application claims the benefit of U.S. Provisional Application No. 63/052,103, filed Jul. 15, 2020, which is incorporated herein by reference.

### SEQUENCE LISTING

The Sequence Listing written in file 559398_SeqListing_ST25.txt is 3 kilobytes in size, was created May 28, 2021, and is hereby incorporated by reference BACKGROUND

Fibroblast growth factor 7 (FGF7, also known as keratinocyte growth factor or KGF) is a ligand for fibroblast growth factor receptor 2 (FGFR2) IIIb isoform that is expressed in many epithelial tissues. Full length human FGF7 consists of 194 amino acids (163 amino acids without the 31 amino acid signal peptide on the N terminus). Previous studies have shown that giving exogenous full length recombinant human KGF (rhKGF) ameliorates toxin or radiation-mediated injury in many epithelial tissues including oral, retinal, lung alveolar, and intestinal epithelia (Dorr et al. 2002, Farrell et al. 2002, Hu et al. 2018, Khan et al. 1997, Wu et al. 1998, and Takeoka et al. 1997). Exogenous rhKGF given subcutaneously prior to cyclophosphamide blocks apoptosis and accelerates repair of bladder urothelium in rodents (Narla et al. 2020 and Ulich et al. 1997). It has been shown that KGF administration leads to higher fidelity urothelial repair after cyclophosphamide injury even 6 months after injury (Narla et al. 2020 and unpublished). Palifermin (brand name Kepivance, marketed by Biovitrum), is a 140 amino acid derivative of human FGF7 that has been approved for clinical use as an intravenous infusion to prevent oral mucosal ulcers in patients receiving chemotherapy and stem cell transplants (for diseases such as Hodgkin's disease, multiple myeloma, and leukemia).

### SUMMARY

Described herein are FGF7-p peptides that bind to and are agonists of FGFR2IIIb. The FGF7-p peptides bind to and activate FGFR2IIIb leading to increased expression of phosphorylated fibroblast growth factor receptor substrate 2 (pFRS2α) and phosphorylated RAC-alpha serine/threonine-protein kinase (pAKT). In some embodiments, an FGF7-p peptide comprises or consists of a peptide having at least 80% amino acid sequence identity to the peptide YASAKWTHNGGEMFVALNQ (SEQ ID NO. 1). In some embodiments, an FGF7-p peptide comprises or consist of a 16-22 amino acid peptide differing by 0, 1, 2, or 3 amino acid substitutions, deletions, insertions, or combinations thereof from SEQ ID NO. 1. The described FGF7-p peptides are easier and cheaper to make than full length FGF7 or palifermin, and are more heat stable than full length FGF7 or palifermin.

Additionally described are compositions comprising one or more of the described FGF7-p peptides. The compositions can contain one or more excipients that increase or modify solubility, delivery, stability, bioavailability, efficacy, pharmacokinetics, or patient acceptability of the FGF7-p peptides. Compositions comprising FGF7-p peptides can be administered to a subject, such as a human or animal subject, for the treatment and/or prevention of symptoms and diseases associated with epithelial damage, including epithelial injury.

The described FGF7-p peptides can be used in methods of therapeutic treatment and/or prevention of symptoms and/or diseases associated with epithelial cell or tissue damage or injury. Symptoms and/or diseases associated with epithelial cell or tissue damage or injury include, but are not limited to, bladder urothelial damage, mucositis, gastrointestinal epithelial damage, lung epithelial damage, retinal epithelial damage, and skin epithelial damage. Such methods comprise administration of one or more of the FGF7-p peptides as described herein to a subject. The subject is administered a therapeutically effective amount of any one or more of the described FGF7-p peptides thereby treating or preventing epithelial damage or injury or one or more symptoms caused by the epithelial damage or injury. The subject can be, but is not limited to, a human subject.

In some embodiments, the described FGF7-p peptides can be used to enhance growth and proliferation of epithelial cells or tissue.

In some embodiments, a therapeutically effective amount of one or more of the described FGF7-p peptides is administered to a subject, thereby activating FGFR2IIIb in the subject. In some embodiments, one or more of the described FGF7-p peptides is used to treat a subject having a disease or disorder that would benefit from activation of the FGFR2IIIb receptor. In some embodiments, one or more of the described FGF7-p peptides is used to treat or prevent at least one symptom in a subject having a disease or disorder that would benefit from activation of the FGFR2IIIb receptor.

In some embodiments, the described FGF7-p peptides can be administered to a subject to inhibit bladder urothelial damage caused by chemotherapy or other toxins, or infection.

In some embodiments, the described FGF7-p peptides can be administered to a subject to treat or inhibit bladder injury associate with bladder pain syndrome.

In some embodiments, the described FGF7-p peptides can be administered to a subject to inhibit bladder urothelial cell apoptosis induced by radiation. In some embodiments, the described FGF7-p peptides can be administered to a subject to inhibit radiation-induced bladder urothelial damage.

### BRIEF DESCRIPTION OF THE DRAWINGS

FIG. 1A-H. Urothelial pFRS2α and pAKT staining in DMSO at 24 hours and FGF7-p (40 mg/kg) at 24, 48 and 72 hours. A-D. Immunofluorescence for pFRS2α (white) is weak and patchy in DMSO and FGF7-p at 24 hours (A, B, arrowheads) with most urothelial regions showing no staining (A, B, arrows). pFRS2α staining is robust and throughout the urothelium at 48 hours (C, arrowheads) and weakens by 72 hours (D, arrowhead). E-H. Immunofluorescence for pAKT shows no urothelial staining in 24 hour DMSO, 24 hour FGF7-p and 48 hour FGF7-p (E-G, arrows), but robust staining throughout the urothelium in 72 hour FGF7-p samples (H, arrowhead). “L”=Lumen. Dotted lines (panels E-G) are at the border of the urothelium and the lumen.

FIG. 2A-E. Comparison of urothelial pAKT staining in 72 hour DMSO, 72 hour FGF7-p and 30, 48 and 96 hour full length FGF7. IF for pAKT (white, arrowheads) shows no staining in the 72 hour DMSO sample (A) but robust staining throughout the urothelium in the 72 hour FGF7-p sample (B). In the full length samples, 30 and 48 hour staining is robust (C, D), although staining does not appear as robust as the 72 hour FGF7-p sample (B). By 96 hours after receiving full length FGF7, pAKT staining is absent (E). “L”=Lumen. “Blood”=artifactual staining from blood. Dotted lines (panels A and E) are at the border of the urothelium and the lumen.

FIG. 3. Immunofluorescence micrographs illustrating protection of urothelium by FGF7-p. FGF7-p blocks cyclophosphamide-induced urothelial apoptosis, likely via phosphorylation of AKT, S6 kinase and BAD. (A-B) TUNEL staining (white) showing sloughing apoptotic urothelial cells (arrow) in DMSO-treated mice (A) and intact and non-apoptotic urothelial cells in FGF7-p-treated mice (B) 24 hours after cyclophosphamide. (C-D) Phospho-AKT (pAKT) staining is weak in DMSO-treated urothelium (C) and strong in FGF7-p-treated urothelium (D) 24 hours after cyclophosphamide injection. (E-F) Phospho-S6 kinase (pS6) urothelial staining is reduced in DMSO-treated mice (E) compared to FGF7-p-treated mice (F) 24 hours after cyclophosphamide injection. (G-H) Phospho-BAD (pBAD) staining is very minimal in DMSO-treated urothelium (G) but strong in FGF7-p-treated urothelium (H). Solid white lines=borders of the urothelium (below the lines) and submucosa (above the lines).

FIG. 4. FGF7p blocks most radiation-induced urothelial apoptosis. TUNEL staining (white) reveals vehicle-treated (DMSO) mice (left panel) have many apoptotic urothelial cells (white) that are sloughing (arrowheads) or attached to the bladder (arrows). FGF7p-treated mice have many regions with almost no (middle panel) or minimal urothelial apoptosis (right panel) (arrows) compared vehicle/DMSO. Solid white lines=borders of the urothelium (above the lines) and submucosa (below the lines).

### DETAILED DESCRIPTION

Before describing the present teachings in detail, it is to be understood that the disclosure is not limited to specific compositions or process steps, as such may vary. As used in this specification and the appended claims, the singular form “a,” “an,” and “the” include plural references unless the context clearly dictates otherwise. Thus, for example, reference to “a peptide” includes a plurality of peptides and the like. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions such as “not including the endpoints”; thus, for example, “within 10-15” includes the values 10 and 15.

The use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

A “polypeptide” or “peptide” is a polymeric form of naturally occurring and/or non-naturally occurring amino acids. The amino acids can be coded or non-coded amino acids, chemically or biochemically modified or derivatized amino acids, or D- or L-form amino acids. The amino acids can be connected by peptide bonds or modified peptide bonds. A peptide is said to have an “N-terminus” and a “C-terminus.” The term “N-terminus” relates to the start of a peptide. An N-terminal amino acid may have a free amine group (—NH₂), or the terminal amine group may be substituted or modified. The term “C-terminus” relates to the end of a peptide. A C-terminal amino acid may have a free carboxyl group (—COOH), or the carboxyl group may be substituted or modified.

A “homologous” sequence (e.g., nucleic acid sequence or amino acid sequence) refers to a sequence that is either identical or substantially similar to a known reference sequence, such that it is, for example, at least 75%, at least 80%, at least 85%, at least 90%, at least 95%, at least 96%, at least 97%, at least 98%, at least 99%, or 100% identical to the known reference sequence. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences.

The term “isolated” means that the material is removed from its original environment (e.g., the natural environment, if it is naturally occurring). For example, a naturally-occurring polynucleotide or polypeptide present in a living animal is not isolated, but the same polynucleotide or polypeptide, separated from some or all of the coexisting materials in the natural system, is isolated.

The terms “treat,” “treatment,” and the like, mean the methods or steps taken to provide relief from or alleviation of the number, severity, and/or frequency of one or more symptoms of a disease or condition in a subject.

### I. FGF7-p Peptides

Described are FGF7-p peptides that bind to or have affinity for FGFR2IIIb and are able to activate FGFR2IIIb. The FGF7-p peptides comprise the amino acid sequence YASAKWTHNGGEMFVALNQ (SEQ ID NO. 1) or amino acid sequences differing from SEQ ID NO: 1 by 0, 1, 2, or 3 amino acids. In some embodiments, the FGF7-p peptides comprise the amino acid sequence YASAKWTHNGGEMFVALNQ (SEQ ID NO. 1) or amino acid sequences differing from SEQ ID NO: 1 by 0, 1, 2, or 3 conservative amino acids changes.

In some embodiments, the FGF7-p peptide comprises or consists of a peptide having at least 80%, at least 85%, or at least 90%, amino acid sequence identity to the peptide YASAKWTHNGGEMFVALNQ (SEQ ID NO. 1).

Described are FGF7-p peptides that bind to or have affinity for FGFR2IIIb and are able to activate FGFR2IIIb. In some embodiments, the FGF7-p peptides are 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 amino acids in length and comprise an amino acid sequence differing by no more than 3 amino acids from YASAKWTHNGGEMFVALNQ (SEQ ID NO. 1). In some embodiments, the FGF7-p peptides are 19, 19-20, 1-21, 19-22, 19-25, 19-30, 19-35, 19-40, 19-45, 19-50, 19-55, 19-60, 19-65, 19-70, 19-75, 19-80, 19-85, 19-90, 19-95, 19-100, 19-105, 19-110, 19-115, 19-120, 19-125, or 19-130 amino acids in length and comprise an amino acid sequence differing by no more than 3 amino acids from YASAKWTHNGGEMFVALNQ (SEQ ID NO. 1). In some embodiments, the FGF7-p peptides are no more than about 19 amino acids, no more than about 20 amino acids, no more than about 21 amino acids, no more than about 22 amino acids, no more than about 23 amino acids, no more than about 24 amino acids, no more than about 25 amino acids, no more than about 30 amino acids, no more than about 35 amino acids, no more than about 40 amino acids, no more than about 45 amino acids, no more than about 50 amino acids, no more than about 55 amino acids, no more than about 60 amino acids, no more than about 65 amino acids, no more than about 70 amino acids, no more than about 75 amino acids, no more than about 80 amino acids, no more than about 85 amino acids, no more than about 90 amino acids, no more than about 95 amino acids, no more than about 100 amino acids, no more than about 105 amino acids, no more than about 110 amino acids, no more than about 115 amino acids, no more than about 120 amino acids, no more than about 125 amino acids, or no more than about 130 amino acids in length and comprise an amino acid sequence differing by no more than 3 amino acids from YASAKWTHNGGEMFVALNQ (SEQ ID NO. 1)

In some embodiments, the FGF7-p peptide consists of 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 (MHKWILTWILPTLLYRSCFHIICLVGTISLACNDMTPEQMATNVNCSSPERHTRSYD YMEGGDIRVRRLFCRTQWYLRIDKRGKVKGTQEMKNNYNIMEIRTVAVGIVAIKGV ESEFYLAMNKEGKLYAKKECNEDCNFKELILENHYNTYASAKWTHNGGEMFVALN QKGIPVRGKKTKKEQKTAHFLPMAIT; UniProtKB—P21781) and comprises the amino acid sequence of SEQ ID NO. 1. In some embodiments, the FGF7-p peptide consists of a sequence having at least 60% homology to 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 and comprises the amino acid sequence of SEQ ID NO. 1. In some embodiments, the FGF7-p peptide consists of a sequence having at least 70% homology to 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 and comprises the amino acid sequence of SEQ ID NO. 1. In some embodiments, the FGF7-p peptide consists of a sequence having at least 75% homology to 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 and comprises the amino acid sequence of SEQ ID NO. 1. In some embodiments, the FGF7-p peptide consists of a sequence having at least 80% homology to 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 and comprises the amino acid sequence of SEQ ID NO. 1. In some embodiments, the FGF7-p peptide consists of a sequence having at least 85% homology to 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 and comprises the amino acid sequence of SEQ ID NO. 1. In some embodiments, the FGF7-p peptide consists of a sequence having at least 90% homology to 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 and comprises the amino acid sequence of SEQ ID NO. 1. In some embodiments, the FGF7-p peptide consists of a sequence having at least 95% homology to 16-25, 16-30, 16-35, 16-40, 16-45, 16-50, 16-55, 16-60, 16-65, 16-70, 16-75, 16-80, 16-85, 16-90, 16-95, 16-100, 16-105, 16-110, 16-115, 16-120, 16-125, or 16-130 contiguous amino acids from SEQ ID NO: 2 and comprises the amino acid sequence of SEQ ID NO. 1.

In some embodiments, an FGF7-p peptide comprises a 16-22 amino acid peptide differing by 0, 1, 2, or 3 amino acids from SEQ ID NO. 1. In some embodiments, an FGF7-p peptide comprises a 16, 17, 18, 19, 20, 21, or 22 amino acid peptide differing by 0, 1, 2, or 3 amino acids from SEQ ID NO. 1. In some embodiments, an FGF7-p peptide comprises a 17-21 amino acid peptide differing by 0, 1, 2, or 3 amino acids from SEQ ID NO. 1. In some embodiments, an FGF7-p peptide comprises a 18-20 amino acid peptide differing by 0, 1, 2, or 3 amino acids from SEQ ID NO. 1. In some embodiments, an FGF7-p peptide comprises a 19 amino acid peptide differing by 0, 1, 2, or 3 amino acids from SEQ ID NO. 1.

In some embodiments, an FGF7-p peptide consists of a 16-22 amino acid peptide differing by 0, 1, 2, or 3 amino acids from SEQ ID NO. 1. In some embodiments, an FGF7-p peptide consists or a 16, 17, 18, 19, 20, 21, or 22 amino acid peptide differing by 0, 1, 2, or 3 amino acids from SEQ ID NO. 1. In some embodiments, an FGF7-p peptide consists of a 17-21 amino acid peptide differing by 0, 1, 2, or 3 amino acids from SEQ ID NO. 1. In some embodiments, an FGF7-p peptide consists of a 18-20 amino acid peptide differing by 0, 1, 2, or 3 amino acids from SEQ ID NO. 1. In some embodiments, an FGF7-p peptide consists of a 19 amino acid peptide differing by 0, 1, 2, or 3 amino acids from SEQ ID NO. 1.

In some embodiments, an FGF7-p peptide comprises a 17-21 amino acid peptide differing by 0, 1, or 2 amino acids from SEQ ID NO. 1. In some embodiments, an FGF7-p peptide comprises a 17, 18, 19, 20, or 21 amino acid peptide differing by 0, 1, or 2 amino acids from SEQ ID NO. 1. In some embodiments, an FGF7-p peptide comprises a 18-20 amino acid peptide differing by 0, 1, or 2 amino acids from SEQ ID NO. 1. In some embodiments, an FGF7-p peptide comprises a 19 amino acid peptide differing by 0, 1, or 2 amino acids from SEQ ID NO. 1.

In some embodiments, an FGF7-p peptide consists of a 17-21 amino acid peptide differing by 0, 1, or 2 amino acids from SEQ ID NO. 1. In some embodiments, an FGF7-p peptide consists or a 17, 18, 19, 20, or 21 amino acid peptide differing by 0, 1, or 2 amino acids from SEQ ID NO. 1. In some embodiments, an FGF7-p peptide consists of a 18-20 amino acid peptide differing by 0, 1, or 2 amino acids from SEQ ID NO. 1. In some embodiments, an FGF7-p peptide consists of a 19 amino acid peptide differing by 0, 1, or 2 amino acids from SEQ ID NO. 1.

In some embodiments, an FGF7-p peptide comprises a 18-20 amino acid peptide differing by 0 or 1 amino acids from SEQ ID NO. 1. In some embodiments, an FGF7-p peptide comprises an 18, 19, or 20 amino acid peptide differing by 0 or 1 amino acids from SEQ ID NO. 1. In some embodiments, an FGF7-p peptide comprises a 19 amino acid peptide differing by 0 or 1 amino acids from SEQ ID NO. 1.

In some embodiments, an FGF7-p peptide consists of a 18-20 amino acid peptide differing by 0 or 1 amino acids from SEQ ID NO. 1. In some embodiments, an FGF7-p peptide consists or an 18, 19, or 20 amino acid peptide differing by 0 or 1 amino acids from SEQ ID NO. 1. In some embodiments, an FGF7-p peptide consists of a 19 amino acid peptide differing by 0 or 1 amino acids from SEQ ID NO. 1.

In some embodiments, an FGF7-p peptide comprises the amino acid sequence of SEQ ID NO: 1. In some embodiments, an FGF7-p peptide comprises a 19 amino acid peptide consisting of the amino acid sequence of SEQ ID NO: 1. In some embodiments, an FGF7-p peptide consists of the amino acid sequence of SEQ ID NO: 1.

In some embodiments, an FGF7-p peptide is 16-22 amino acids in length and comprises 19 contiguous amino acids having at least 80%, at least 85%, or at least 90% sequence identity to the amino acid sequence of SEQ ID NO: 1.

In some embodiments, an FGF7-p peptide is 16-22 amino acids in length and consists of 19 contiguous amino acids having at least 80%, at least 85%, or at least 90% sequence identity to the amino acid sequence of SEQ ID NO: 1.

In some embodiments, an FGF7-p peptide is 17-21 amino acids in length and comprises 19 contiguous amino acids having at least 80%, at least 85%, or at least 90% sequence identity to the amino acid sequence of SEQ ID NO: 1.

In some embodiments, an FGF7-p peptide is 17-21 amino acids in length and consists of 19 contiguous amino acids having at least 80%, at least 85%, or at least 90% sequence identity to the amino acid sequence of SEQ ID NO: 1.

In some embodiments, an FGF7-p peptide is 18-20 amino acids in length and comprises 19 contiguous amino acids having at least 80%, at least 85%, or at least 90% sequence identity to the amino acid sequence of SEQ ID NO: 1.

In some embodiments, an FGF7-p peptide is 18-20 amino acids in length and consists of 19 contiguous amino acids having at least 80%, at least 85%, or at least 90% sequence identity to the amino acid sequence of SEQ ID NO: 1.

In some embodiments, an FGF7-p peptide is 19 amino acids in length and comprises 19 contiguous amino acids having at least 80%, at least 85%, or at least 90% sequence identity to the amino acid sequence of SEQ ID NO: 1.

In some embodiments, an FGF7-p peptide is 19 amino acids in length and consists of 19 contiguous amino acids having at least 80%, at least 85%, or at least 90% sequence identity to the amino acid sequence of SEQ ID NO: 1.

In some embodiments, an FGF7-p peptide can have 0, 1, 2, or 3 amino acid residue substitutions, insertions, or deletions compared to the amino acid sequence of SEQ ID NO: 1. Peptide variants and derivatives are well understood to those of skill in the art and can involve amino acid sequence modifications. An amino acid sequence modification can be a substitution, insertion, or deletion. Insertions include amino and/or carboxyl terminal additions as well as intrasequence insertions of single or multiple amino acid residues. Deletions include the removal of one or more amino acid residues from the peptide sequence. Substitutions include substitution of an amino acid residue at a given position in the amino acid sequence with a different amino acid. Insertions, deletions, and substitutions can occur at a single position or multiple positions. Insertions, deletions, and substitutions can occur at adjacent positions and/or non-adjacent positions. In some embodiments the one or more of the substitutions is a conservative amino acid substitution. Substitutions, deletions, insertions, or any combination thereof may be combined to arrive at a final FGF7-p peptide. For a peptide differing by 0, 1, 2, or 3 amino acids from a reference sequence, the peptide can have substitutions, insertions, or deletions of 0, 1, 2, or 3 amino acids in any combination or order.

A “conservative amino acid substitution” is a substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of Sa non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acids.

| | | | | | | Exemplary |
|---|---|---|---|---|---|---|
| | | | | | | Conservative |
| Amino acid | Abbreviations | | Polarity | Charge | Hydrophobicity | Substitution |
| Alanine | Ala | A | Nonpolar | Neutral | 1.8 | Ser |
| Arginine | Arg | R | Polar | Positive | −4.5 | Lys, Gln |
| Asparagine | Asn | N | Polar | Neutral | −3.5 | Gln, His |
| Aspartic acid | Asp | D | Polar | Negative | −3.5 | Glu |
| Cysteine | Cys | C | Nonpolar | Neutral | 2.5 | Ser |
| Glutamic acid | Glu | E | Polar | Negative | −3.5 | Asn, Lys |
| Glutamine | Gln | Q | Polar | Neutral | −3.5 | Asp |
| Glycine | Gly | G | Nonpolar | Neutral | −0.4 | Pro |
| Histidine | His | H | Polar | Positive | −3.2 | Asn, Gln |
| Isoleucine | Ile | I | Nonpolar | Neutral | 4.5 | Leu, Val |
| Leucine | Leu | L | Nonpolar | Neutral | 3.8 | Ile, Val |
| Lysine | Lys | K | Polar | Positive | −3.9 | Arg, Gln |
| Methionine | Met | M | Nonpolar | Neutral | 1.9 | Leu, Ile |
| Phenylalanine | Phe | F | Nonpolar | Neutral | 2.8 | Met, Leu, Tyr |
| Proline | Pro | P | Nonpolar | Neutral | −1.6 | |
| Serine | Ser | S | Polar | Neutral | −0.8 | Thr |
| Threonine | Thr | T | Polar | Neutral | −0.7 | Ser |
| Tryptophan | Trp | W | Nonpolar | Neutral | −0.9 | Tyr |
| Tyrosine | Tyr | Y | Polar | Neutral | −1.3 | Trp, Phe |
| Valine | Val | V | Nonpolar | Neutral | 4.2 | Ile, Leu |

In some embodiments, an FGF7-p peptide contains one or more L-amino acids. In some embodiments, all of the amino acids in an FGF7-p peptide are L amino acids. In some embodiments, 1, 2, 3, 4, 5, 6, 7, 8, 9, 10, 11, 12, 13, 14, 15, 16, 17, 18, 19, 20, 21, or 22 of the amino acids in an FGF7-p peptide are L amino acids. In some embodiments, an FGF7-p peptide contains one or more D-amino acids. In some embodiments, all of the amino acids in an FGF7-p peptide are D amino acids. In some embodiments, 1, 2, 3, 4, 5, 6, 7, 8, 9, 10, 11, 12, 13, 14, 15, 16, 17, 18, 19, 20, 21, or 22 of the amino acids in an FGF7-p peptide are D amino acids. D-amino acids can be used to generate more stable peptides.

In some embodiments, the peptide contains one or more amino acid analogs. Amino analogs can alter chemical stability, pharmacological properties (half-life, absorption, potency, efficacy, etc.), specificity, or antigenicity of a peptide.

Binding of an FGF7-p peptide to FGFR2IIIb can be determined by methods known in the art for analyzing binding of one protein or peptide to another peptide or protein, such as immunoprecipitation. In some embodiments, the described FGF7-p peptides compete with full length FGF7 or Palifermin for binding to FGFR2IIIb. In some embodiments, an FGF7-p peptide binding to FGFR2IIIb is at least 10%, at least 15%, at least 20%, or at least 25% of full-length FGF7 binding to FGFR2IIIb as measured by the same assay.

Activation of FGFR2IIIb can be determined by induction or increase in expression of phosphorylated fibroblast growth factor receptor substrate 2 (pFRS2α) and/or phosphorylated RAC-alpha serine/threonine-protein kinase (pAKT) in epithelial cells or tissue. Induction of pFRS2α or pAKT can be determined by measuring pFRS2α or pAKT protein levels in epithelial cells or tissue after exposure of the epithelial cells or tissue to an effective dose of FGF7-p peptide. pFRS2α and pAKT protein levels can be determined by methods known in the art. An increase in expression of pFRS2α and pAKT in epithelial cells or tissue following exposure of the epithelial cells or tissue to an effective dose of FGF7-p peptide indicates activation of FGFR2IIIb. In some embodiments, induction of pFRS2α or pAKT can be determined by immunostaining in urothelial tissue 1, 2, 3, 4, 5, 6, or 7 days after administering an effective does of FGF7-p peptide to a subject, such as a model organism (e.g., mouse).

In some embodiments, the FGF7-p peptide is resistant to heat. In some embodiments, the FGF7-p peptide is more resistant to heat that full length FGF7. In some embodiments, the FGF7-p peptide can be sterilized by heat, such as by boiling.

The described FGF7-p peptides can be chemically synthesized. The described FGF7-p peptides can be prepared using any of a number of chemical peptide synthesis techniques known in the art. The peptides can be synthesized using solution methods or solid phase methods. Solid phase synthesis in which the C-terminal amino acid of the polypeptide sequence is attached to an insoluble support followed by sequential addition of the remaining amino acids in the sequence is one synthetic method for preparing the polypeptides. Alternatively, the described FGF7-p peptides may be prepared by biosynthesis in a host cell. For biosynthesis in a host cell, a nucleic acid encoding the FGF7-p peptide is created and transformed or transfected into the host cell. In some embodiments, the host cell is an industrially scalable microorganism. In some embodiments, an FGF7-p peptide is isolated and/or purified. The peptides can be isolated and purified from the synthesis reaction mixture by means of peptide purification known to in the art. For example, the peptides may be purified using known chromatographic procedures such as reverse phase HPLC, gel permeation, ion exchange, size exclusion, affinity, partition, or countercurrent distribution.

Any of the described FGF7-p peptides can be provided in an aqueous solution or as a lyophilized powder. Any of the described FGF7-p peptides can be for use in preparation of a medicament.

### II. Compositions Comprising an FGF7-p Peptide

In some embodiments, an FGF7-p peptide is combined with one or more pharmaceutically acceptable excipients, including but no limited to, vehicles, carriers, diluents, and/or delivery polymers, thereby forming a composition. The composition may be a pharmaceutical composition suitable for in vivo delivery to a subject. The subjection can be a mammal. The mammal can be, but is not limited to, a human, a dog, a cat, a rabbit, a pig, a rat, a guinea pig, or a mouse.

A pharmaceutical composition or medicament includes a pharmacologically effective amount of at least one of the described FGF7-p peptides and optionally one or more pharmaceutically acceptable excipients. Pharmaceutically acceptable excipients (excipients) are substances other than the Active Pharmaceutical ingredient (API, therapeutic product, e.g., FGF7-p peptide) that are intentionally included in the drug delivery system. Excipients do not exert or are not intended to exert a therapeutic effect at the intended dosage. Excipients may act to a) aid in processing of the drug delivery system during manufacture; b) protect, support or enhance stability, bioavailability or patient acceptability of the API; c) assist in product identification; and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions can contain other additional components commonly found in pharmaceutical compositions. Such additional components can include, but are not limited to, anti-pruritics, astringents, local anesthetics, or anti-inflammatory agents (e.g., antihistamine, diphenhydramine, etc.).

The carrier can be, but is not limited to, a solvent or dispersion medium containing, for example, water, ethanol, polyol (for example, glycerol, propylene glycol, and liquid polyethylene glycol), and suitable mixtures thereof. A carrier may also contain adjuvants such as preservatives, wetting agents, emulsifying agents and dispersing agents. A carrier may also contain isotonic agents, such as sugars, polyalcohols, sodium chloride, and the like into the compositions.

Pharmaceutically acceptable refers to those properties and/or substances which are acceptable to the subject from a pharmacological/toxicological point of view. The phrase pharmaceutically acceptable refers to molecular entities, compositions, and properties that are physiologically tolerable and do not typically produce an allergic or other untoward or toxic reaction when administered to a subject. In some embodiments, a pharmaceutically acceptable compound is approved by a regulatory agency of the Federal or a state government or listed in the U.S. Pharmacopeia or other generally recognized pharmacopeia for use in animals and more particularly in humans.

In some embodiments, a described FGF7-p peptide or composition containing the FGF7-p peptide is formulated in 0.1-10% dimethyl sulfoxide (DMSO). In some embodiments, a described FGF7-p peptide or composition containing the FGF7-p peptide is formulated in 0.1, 0.2, 0.3, 0.4, 0.5, 0.6, 0.7, 0.8, 0.9, 1, 2, 3, 4, 5, 6, 7, 8, 9, or 10% DMSO. In some embodiments, a described FGF7-p peptide or composition containing the FGF7-p peptide is formulated in phosphate buffered saline. In some embodiments, a described FGF7-p peptide or composition containing the FGF7-p peptide is formulated in HEPES (N-2-hydroxyethylpiperazine-N′-2-ethanesulfonic acid) buffer.

Any of the described compositions can be provided as an aqueous solution or as a lyophilized powder. Any of the described compositions can be for use in preparation of a medicament.

### III. Combinations

Any of the described FGF7-p peptides can be combined with one or more additional therapeutic agents for use in providing a therapeutic effect in a subject.

In some embodiments, an FGF7-p peptide is used in combination with one or more other therapies known in the art to prevent, treat, or ameliorate one or more symptoms associated with epithelial cell or tissue damage or injury.

In some embodiments, an FGF7-p peptide is combined an agent that reduces or decreases the risk of bleeding from the bladder, reduces the incidence of hemorrhagic cystitis or hematuria, and/or binds to acrolein, such as, but not limited to, mesna. In some embodiments, an FGF7-p peptide is combined with an anti-inflammatory or anti-oxidant.

In some embodiments, administration of FGF7-p peptide is combined with chondroitin sulfate or formalin instillation into the bladder, hydration, and/or hyperbaric oxygen therapy in treat bladder injury resulting from radiation.

In some embodiments, administration of FGF7-p peptide is combined with antibiotics and/or antiviral therapies in the treatment of bladder and other epithelial tissue infection.

In some embodiments, administration of FGF7-p is combined with intermittent bladder catherization, anti-cholinergic medications and/or botulinum A toxin injection directly into the bladder in the treatment of spinal cord injury, neurogenic bladder, and non-neurogenic neurogenic bladder.

In some embodiments, administration of FGF7-p is combined with combination with one or more non-steroidal anti-inflammatory medication and/or tricyclic antidepressants in the treatment of bladder pain syndrome.

### IV. Methods of Use

The described FGF7-p peptides and compositions containing any of the described FGF7-p peptides can be used to provide methods for the therapeutic treatment of diseases. Such methods include administration of a pharmaceutical composition described herein to a subject.

In some embodiments, a described FGF7-p peptide or composition containing the FGF7-p peptide can be used for the treatment of epithelial damage or injury in a subject. In some embodiments, a described FGF7-p peptide or composition containing the FGF7-p peptide can be used in the treatment of a subject at risk of developing epithelial damage or injury. In some embodiments, a described FGF7-p peptide or composition containing the FGF7-p peptide is used to treat, prevent, or manage a clinical presentation of epithelial damage or injury. In some embodiments, administration of a described FGF7-p peptide or composition containing the FGF7-p peptide can be used to decrease the number, severity, and/or duration of epithelial damage or injury. In some embodiments, administration of a described FGF7-p peptide or composition containing the FGF7-p peptide can be used to decrease the chance of developing epithelial damage or injury. In some embodiments, the methods include administering a described FGF7-p peptide or composition containing the FGF7-p peptide to a subject to be treated. In some embodiments, a therapeutically or prophylactically effective amount of one or more FGF7-p peptides or a composition containing the one or more FGF7-p peptides is administered to a subject in need of such treatment, prevention or management. In some embodiments, the subject is a mammal, including, but not limited to, a human patient. The subject may be an adult, adolescent, child, or infant.

The epithelial damage or injury can be caused by, for example, chemotherapy, chemicals toxins (including toxic metabolites from drugs), radiation (including radiation therapy), infection (included bacterial infection and viral infection). The epithelial damage or injury can be in, for example, bladder, oral mucosa, skin, gastrointestinal epithelia (including mouth, stomach, and intestine), lung (alveolar) airway epithelia, and retinal epithelia.

In some embodiments, the subject is administered a therapeutically effective amount of one or more of the described FGF7-p peptides or a composition containing the one or more FGF7-p peptides, thereby treating, preventing, or managing at least one symptom associated with damage or injury to epithelial tissue. The epithelial tissue can be, but is not limited to, bladder, oral mucosa, skin, gastrointestinal epithelia (including mouth, stomach, and intestine), lung (alveolar) airway epithelia, and retinal epithelia.

In some embodiments, a described FGF7-p peptide or composition containing the FGF7-p peptide can be used to facilitate healing of epithelial tissue in a subject. In some embodiments, administration of a described FGF7-p peptide or composition containing the FGF7-p peptide can be used to block apoptosis in epithelial tissue, accelerate repair of epithelial tissue (including, but not limited to, bladder urothelium), stimulate growth or development of epithelial tissue, or increase or accelerate wound healing in epithelial tissues. In some embodiments, the methods include administering a described FGF7-p peptide or composition containing the FGF7-p peptide to a subject to be treated. In some embodiments, a therapeutically effective amount of one or more FGF7-p peptides or a composition containing the one or more FGF7-p peptides is administered to a subject in need of such treatment, prevention or management. In some embodiments, the subject is a mammal, including, but not limited to, a human patient. The subject may be an adult, adolescent, child, or infant. The epithelial tissue can be, but is not limited to, bladder epithelia, oral mucosa, skin, gastrointestinal epithelia (including mouth, stomach, and intestine, lung (alveolar) airway epithelia, and retinal epithelia.

In some embodiments, a described FGF7-p peptide or composition containing the FGF7-p peptide can be used to treat a subject having bladder urothelial damage or bladder injury. In some embodiments, a described FGF7-p peptide or composition containing the FGF7-p peptide can be used to treat a subject at risk of developing bladder urothelial damage bladder injury. In some embodiments, a described FGF7-p peptide or composition containing the FGF7-p peptide is used to treat, prevent, or manage a clinical presentation of bladder urothelial damage or bladder injury. In some embodiments, administration of a described FGF7-p peptide or composition containing the FGF7-p peptide can be used to decrease the number, severity, and/or duration of bladder urothelial damage bladder injury. In some embodiments, administration of a described FGF7-p peptide or composition containing the FGF7-p peptide can be used to decrease the chance of developing bladder urothelial damage bladder injury. In some embodiments, the methods include administering a described FGF7-p peptide or composition containing the FGF7-p peptide to a subject to be treated. In some embodiments, a therapeutically or prophylactically effective amount of one or more FGF7-p peptides or a composition containing the one or more FGF7-p peptides is administered to a subject in need of such treatment, prevention, or management. In some embodiments, the subject is a mammal, including, but not limited to, a human patient. The subject may be an adult, adolescent, child, or infant.

The bladder urothelial damage can be caused by acrolein (a bladder toxic metabolite from cyclophosphamide or ifosfamide, chemical toxins (including toxic metabolites from drugs), radiation (including radiation therapy), infection (included bacterial infection and viral infection), spinal cord injury, neurogenic, or non-neurogenic neurogenic bladder. Bladder injury can be associated with bladder pain syndrome (formerly known as interstitial cystitis).

In some embodiments, a described FGF7-p peptide or composition containing the FGF7-p peptide can be used to treat a subject having mucositis or severe mucositis. In some embodiments, a described FGF7-p peptide or composition containing the FGF7-p peptide can be used to treat a subject at risk of developing mucositis or severe mucositis. In some embodiments, a described FGF7-p peptide or composition containing the FGF7-p peptide is used to treat, prevent, or manage a clinical presentation of mucositis or severe mucositis. In some embodiments, administration of a described FGF7-p peptide or composition containing the FGF7-p peptide can be used to decrease severity, and/or duration of mucositis or severe mucositis. In some embodiments, administration of a described FGF7-p peptide or composition containing the FGF7-p peptide can be used to decrease the chance of developing mucositis or severe mucositis. In some embodiments, the methods include administering a described FGF7-p peptide or composition containing the FGF7-p peptide to a subject to be treated. In some embodiments, a therapeutically or prophylactically effective amount of one or more FGF7-p peptides or a composition containing the one or more FGF7-p peptides is administered to a subject in need of such treatment, prevention, or management. In some embodiments, the subject is a mammal, including, but not limited to, a human patient. The subject may be an adult, adolescent, child, or infant. The mucositis or severe mucositis can be caused by, for example, chemotherapy, chemicals toxins (including toxic metabolites from drugs), radiation (including radiation therapy), epithelial infection (included bacterial infection and viral infection).

The mucositis or severe mucositis can be, but is not limited to oral mucositis. The subject can be, but is not limited to, a patient this is receiving or will receive chemotherapy or radiation therapy, or undergoing autologous or allogeneic transplant. The subject undergoing autologous or allogeneic transplant can be a patient undergoing bone marrow or stem cell transplant. The subject undergoing autologous or allogeneic transplant can be a subject with hematologic malignancy receiving myelotoxic therapy requiring hematopoietic stem cell support. The hematologic malignancy can be, but is not limited to, blood cancer, leukemia, lymphoma, and lymphoblastic leukemia. The subject receiving chemotherapy or radiation therapy can be, but is not limited to, a patient having sarcoma, metastatic colorectal cancer, or head and neck cancer.

In some embodiments, the described FGF7-p peptides or compositions containing the FGF7-p peptides can be used to decrease the incidence and/or duration of severe oral mucositis in patients with hematologic malignancies receiving myelotoxic therapy.

In some embodiments, the FGF7-p peptide can be used to accelerate immune reconstitution and/or inhibit of graft-versus-host disease in patients undergoing allogeneic transplantation. The allogeneic transplantation ca be, but is not limited to hematopoietic stem cell transplantation.

In some embodiments, the FGF7-p peptide can be used to mitigate dysphagia in lung cancer patients treated with concurrent chemotherapy and/or radiation therapy.

In some embodiments, epithelial damage in a subject to whom a described FGF7-p peptide or composition containing the FGF7-p peptide is administered is reduced by at least about 5%, for example, but at least about 10%, 15%, 20%, 25%, 30%, 35%, 40%, 45%, 50%, 55%, 60%, 65%, 70%, 75%, 80%, 85%, 90%, 95%, or 98% relative to the subject not receiving the FGF7-p peptide or composition containing the FGF7-p peptide. In some embodiments, endothelial repair in a subject to whom a described FGF7-p peptide or composition containing the FGF7-p peptide is administered is increased by at least about 5%, for example, but at least about 10%, 15%, 20%, 25%, 30%, 35%, 40%, 45%, 50%, 55%, 60%, 65%, 70%, 75%, 80%, 85%, 90%, 95%, or 98% relative to the subject not receiving the FGF7-p peptide or composition containing the FGF7-p peptide. Reduction in epithelial damage or an increase in epithelial repair can be assessed by any methods known in the art.

Described are methods of activating FGFR2IIIb in an epithelial cell or tissue, comprising contacting the epithelial cell or tissue with any of the described FGF7-p peptides or compositions containing FGF7-p peptides.

Described are methods of increasing expression of phosphorylated fibroblast growth factor receptor substrate 2 (pFRS2α) in an epithelial cell or tissue comprising contacting the epithelial cell or tissue with any of the described FGF7-p peptides or compositions containing FGF7-p peptides.

Described are methods of increasing expression of phosphorylated RAC-alpha serine/threonine-protein kinase (pAKT) in an epithelial cell or tissue comprising contacting the epithelial cell or tissue with any of the described FGF7-p peptides or compositions containing FGF7-p peptides.

### V. Administration

In some embodiments, a described FGF7-p peptide or compositions containing the FGF7-p peptide can be formulated for administration to a subject. The described FGF7-p peptides can be administered by methods known in the art. A route of administration is the path by which the FGF7-p peptide or a composition containing the FGF7-p peptide is brought into contact with the body. In general, methods of administering drugs and nucleic acids for treatment of a mammal are well known in the art and can be applied to administration of the described FGF7-p peptides and compositions. The described FGF7-p peptides and compositions can be administered via any suitable route in a preparation or formulation appropriately tailored to that route. In general, any suitable method recognized in the art for delivering a peptide can be adapted for use with a herein described FGF7-p peptides and compositions. Routes of administration include, but are not limited to, parenteral, local, direct injection, intraparenchymal, intramuscular, implantation, topical, systemic, intravascular, intravenous, intra-arterial, intraventricular, intralymphatic, transdermal, intracutaneous, intradermal, subdermal, subcutaneous, intraperitoneal, intravesical, intracranial, subdural, intrathecal, epidural, rectal, airway (aerosol), nasal, oral, buccal (mouth/cheek), and sublingual (under the tongue) administration. In some embodiments, the described FGF7-p peptides or compositions are administered by subcutaneous, intravenous, intramuscular, topical, oral, intravesical, intraperitoneal, aerosol, or intrathecal administration.

The described FGF7-p peptides and compositions may be delivered for research purposes or to produce a change in a cell, tissue, organ of subject that is therapeutic.

A “pharmacologically effective amount,” “therapeutically effective amount,” “effective amount,” or “effective dose” refers to that amount of FGF7-p peptide to produce an intended pharmacological, therapeutic, or preventive result.

The described FGF7-p peptides and compositions containing the FGF7-p peptides can be administered to a subject, before developing or after onset of epithelial damage or injury. The described FGF7-p peptides and compositions containing the FGF7-p peptides can be administered to a subject prior to starting chemotherapy or radiation therapy, concurrently with administration of chemotherapy or radiation therapy, after chemotherapy or radiation therapy, or a combination thereof. In some embodiments, an FGF7-p peptide and composition containing the FGF7-p peptide is administered prior to a treatment known to cause epithelial damage. In some embodiments, an FGF7-p peptide or composition containing the FGF7-p peptide is administered after a treatment known to cause epithelial damage. In some embodiments, an FGF7-p peptide or composition containing the FGF7-p peptide is administered concurrently with a treatment known to cause epithelial damage. In some embodiments, an FGF7-p peptide or composition containing the FGF7-p peptide is administered after a treatment known to cause epithelial damage but before onset of epithelial damage or injury.

In some embodiments, a described FGF7-p peptide or composition containing the FGF7-p peptide is administered to a subject starting up to 7 days prior to exposure of the subject to one or more therapies known to cause epithelial damage. In some embodiments, a described FGF7-p peptide or composition containing the FGF7-p peptide is administered to a subject starting up to 1, 2, 3, 4, 5, 6, or 7 days prior to exposure of the subject to one or more therapies known to cause epithelial damage. In some embodiments, a described FGF7-p peptide or composition containing the FGF7-p peptide is administered to a subject starting up to 7 days prior to and concurrent with exposure of the subject to one or more therapies known to cause epithelial damage. In some embodiments, a described FGF7-p peptide or composition containing the FGF7-p peptide is administered to a subject starting up to 1, 2, 3, 4, 5, 6, or 7 days prior to and concurrent with exposure of the subject to one or more therapies known to cause epithelial damage. In some embodiments, a described FGF7-p peptide or composition containing the FGF7-p peptide is administered to a subject starting up to 2 days prior to exposure of the subject to one or more therapies known to cause epithelial damage.

In some embodiments, a described FGF7-p peptide or composition containing the FGF7-p peptide is administered to a subject for up to 7 days after exposure of the subject to one or more therapies known to cause epithelial damage. In some embodiments, a described FGF7-p peptide or composition containing the FGF7-p peptide is administered to a subject for up to 1, 2, 3, 4, 5, 6, or 7 days after exposure of the subject to one or more therapies known to cause epithelial damage. In some embodiments, a described FGF7-p peptide or composition containing the FGF7-p peptide is administered to a subject concurrent with and for up to 7 days after exposure of the subject to one or more therapies known to cause epithelial damage. In some embodiments, the described FGF7-p peptide or composition containing the FGF7-p peptide is administered to a subject concurrent with and for up to 1, 2, 3, 4, 5, 6, or 7 days after exposure of the subject to one or more therapies known to cause epithelial damage.

In some embodiments, a described FGF7-p peptide or composition containing the FGF7-p peptide is administered to a subject starting up to 7 days prior to exposure of the subject to one or more therapies known to cause epithelial damage and up to 7 days after exposure of the subject to one or more therapies known to cause epithelial damage. In some embodiments, a described FGF7-p peptide or composition containing the FGF7-p peptide is administered to a subject starting up to 1, 2, 3, 4, 5, 6, or 7 days prior to exposure of the subject to one or more therapies known to cause epithelial damage and up to 1, 2, 3, 4, 5, 6, or 7 days after exposure of the subject to one or more therapies known to cause epithelial damage. In some embodiments, a described FGF7-p peptide or composition containing the FGF7-p peptide is administered to a subject starting up to 7 days prior to, concurrent with, and up to 7 days after exposure of the subject to one or more therapies known to cause epithelial damage. In some embodiments, a described FGF7-p peptide or composition containing the FGF7-p peptide is administered to a subject starting up to 1, 2, 3, 4, 5, 6, or 7 days prior to, concurrent with, and up to 1, 2, 3, 4, 5, 6, or 7 days after exposure of the subject to one or more therapies known to cause epithelial damage.

In some embodiments, a described FGF7-p peptide or composition containing the FGF7-p peptide is administered to a subject starting at diagnosis or suspected diagnosis of epithelial bacterial or viral infection. The described FGF7-p peptide or composition containing the FGF7-p peptide can be administered for up to 7 days after diagnosis or suspected diagnosis of epithelial bacterial or viral infection or for as long as symptoms persists that may benefit from treatment with FGF7-p peptide. Symptoms that may benefit from treatment with FGF7-p peptide include, but are not limited to, worsening bladder and/or kidney function, worsening obstruction, infection, and decreased glomerular filtration rate.

In some embodiments, a described FGF7-p peptide or composition containing the FGF7-p peptide is administered to a subject starting with onset of spinal cord injury, diagnosis of spinal cord injury, diagnosis of neurogenic bladder, or diagnosis of non-neurogenic neurogenic bladder. The described FGF7-p peptide or composition containing the FGF7-p peptide can be administered for up to 7 days after onset of spinal cord injury, diagnosis of spinal cord injury, diagnosis of neurogenic bladder, or diagnosis of non-neurogenic neurogenic bladder or for as long as symptoms persist that my benefit from treatment with FGF7-p peptide. Symptoms that may benefit from treatment with FGF7-p peptide include, but are not limited to, worsening bladder and/or kidney function, worsening obstruction, infection, and decreased glomerular filtration rate.

In some embodiments, a described FGF7-p peptide or composition containing the FGF7-p peptide is administered to a subject starting with diagnosis of bladder pain syndrome. The described FGF7-p peptide or composition containing the FGF7-p peptide can be administered for up to 7 days after diagnosis bladder pain syndrome or for as long as symptoms persists that may benefit from treatment with FGF7-p peptide. Symptoms that may benefit from treatment with FGF7-p peptide include, but are not limited to, worsening bladder and/or kidney function, worsening obstruction, infection, decreased glomerular filtration rate, and an episode of breakthrough bladder pain.

A described FGF7-p peptide or composition containing the FGF7-p peptide may be administered daily, every two days, every three days, every four days, every five days, every six days, or weekly.

In some embodiments, a described FGF7-p peptide or composition containing the FGF7-p peptide is administered daily, every two day, every three days, every four days, every five days, every six days, or weekly, for as long as symptoms persists that my benefit from treatment with FGF7-p peptide.

### VI. Listing of Embodiments

1. An FGFR2IIIb agonist comprising an FGF7-p peptide, wherein the FGF7-p peptide is no more than about 130 amino acids in length and comprises an amino acid sequence differing by 0, 1, 2, or 3 amino acid substitutions, deletions, insertions, or combinations thereof from the amino acid sequence of SEQ ID NO. 1.
2. An FGFR2IIIb agonist comprising an FGF7-p peptide, wherein the FGF7-p peptide consists of a peptide differing by 0, 1, 2, or 3 amino acid substitutions, deletions, insertions, or combinations thereof from the amino acid sequence of SEQ ID NO. 1.
3. The FGFR2IIIb agonist of embodiment 1, wherein the FGF7-p peptide consists of a peptide differing by 3 amino acid substitutions, deletions, insertions, or combinations thereof from the amino acid sequence of SEQ ID NO. 1.
4. The FGFR2IIIb agonist of embodiment 1, wherein the FGF7-p peptide consists of a peptide differing by 2 amino acid substitutions, deletions, insertions, or combinations thereof from the amino acid sequence of SEQ ID NO. 1.
5. The FGFR2IIIb agonist of embodiment 1, wherein the FGF7-p peptide consists of a peptide differing by 1 amino acid substitution, deletion, or insertion from the amino acid sequence of SEQ ID NO. 1
6. The FGFR2IIIb agonist of embodiment 1, wherein the FGF7-p peptide consists of a peptide having the amino acid sequence of SEQ ID NO. 1.
7. The FGFR2IIIb agonist of any one of embodiments 1-5, wherein the 1, 2, or 3 amino acid substitutions are conservative amino acid substitutions.
8. A composition comprising the FGFR2IIIb agonist of any one of embodiments 1-7.
9. The composition of embodiment 8, further comprising one or more pharmaceutically acceptable excipients.
10. The composition of embodiment 9, wherein at least one of the one or more pharmaceutically acceptable excipients is dimethyl sulfoxide (DMSO).
11. The composition any one of embodiments 8-10, wherein the composition is an aqueous solution.
12. The composition of embodiment 11, wherein the aqueous solution is 0.1% to 10% DMSO.
13. The composition of embodiment 12, wherein the aqueous solution is 10% DMSO.
14. The composition of embodiment 8, wherein the composition is a lyophilized powder.
15. A nucleic acid encoding the FGF7-p peptide of any one of embodiments 1-7.
16. A method of treating a subject having epithelial damage or at risk of developing epithelial damage, comprising administering to the subject a pharmaceutically effective dose of the FGFR2IIIb agonist of any one of embodiments 1-7 or the composition of any one of embodiments 8-14.
17. The method of embodiment 16, wherein the subject has or is at risk of developing bladder urothelial damage, mucositis, gastrointestinal epithelial damage, lung epithelial damage, retinal epithelial damage, or skin epithelial damage.
18. The method of embodiment 15 or 16, wherein the subject has been administered or will be administered chemotherapy or radiation therapy or is undergoing autologous or allogeneic transplant.
19. The method of embodiment 18, wherein the subject has sarcoma, metastatic colorectal cancer, or head and neck cancer
20. The method of embodiment 18, wherein the chemotherapy comprises cyclophosphamide or ifosfamide chemotherapy.
21. The method of embodiment 16, wherein the subject has or is a risk of developing bladder pain syndrome.
22. The method of embodiment 16 or 17, wherein the subject has hematologic malignancies and/or is receiving myelotoxic therapy.
23. The method of any one of embodiments 16-22, wherein the subject has or is a risk of developing mucositis, severe mucositis, or severe oral mucositis.
24. The method of any one of embodiments 16-23, wherein the FGFR2IIIb agonist or the composition is administered by parenteral, local, direct injection, intraparenchymal, intramuscular, implantation, topical, systemic, intravascular, intravenous, intra-arterial, intraventricular, intralymphatic, transdermal, intracutaneous, intradermal, subdermal, subcutaneous, intraperitoneal, intravesical, intracranial, subdural, intrathecal, epidural, rectal, airway, nasal, oral, buccal, or sublingual administration.
25. The method of any one of embodiments 16-24, wherein the FGFR2IIIb agonist or the composition is administered prior to, concurrent with, and/or subsequent to onset or diagnosis of epithelial damage.
26. The method of any one of embodiments 16-22, wherein the FGFR2IIIb or the composition is administered prior to, concurrent with, and/or subsequent to administration to the patient of a therapy known to cause epithelial damage.
27. The method of any one of embodiments 16-22, wherein the FGFR2IIIb or the composition is administered prior to, concurrent with, and/or subsequent to onset or diagnosis of spinal cord injury, neurogenic bladder, non-neurogenic neurogenic bladder, or bladder pain syndrome.
28. A method of activating FGFR2IIIb in an epithelial cell or tissue, comprising contacting the epithelial cell or tissue with the FGFR2IIIb agonist of any one of embodiments 1-7 or the composition of any one of embodiments 8-14.
29. A method of increasing expression of phosphorylated fibroblast growth factor receptor substrate 2 (pFRS2α) in an epithelial cell or tissue comprising contacting the epithelial cell or tissue with the FGFR2IIIb agonist of any one of embodiments 1-7 or the composition of any one of embodiments 8-14.
30. A method of increasing expression of phosphorylated RAC-alpha serine/threonine-protein kinase (pAKT) in an epithelial cell or tissue comprising contacting the epithelial cell or tissue with the FGFR2IIIb agonist of any one of embodiments 1-7 or the composition of any one of embodiments 8-14.
31. A method of increasing growth and proliferation of epithelial cells or tissue comprising contacting the epithelial cell or tissue with the FGFR2IIIb agonist of any one of embodiments 1-7 or the composition of any one of embodiments 8-14.

### REFERENCES

Dorr W et al. “The effect of keratinocyte growth factor on healing of manifest radiation ulcers in mouse tongue epithelium.” Cell Prolif 35 Suppl 1, 86-92 (2002).
Farrell C L et al. “The effects of keratinocyte growth factor in preclinical models of mucositis.” Cell Prolif 35 Suppl 1, 78-85 (2002).
Hu H. et al. “Activation of KGFR-Akt-mTOR-Nrf2 signaling protects human retinal pigment epithelium cells from Ultra-violet.” Biochem Biophys Res Commun 495, 2171-2177 (2018).
Khan W B et al. “Enhancement of murine intestinal stem cell survival after irradiation by keratinocyte growth factor.” Radiat Res 148, 248-253 (1997).
Wu K I et al. “Keratinocyte growth factor promotes alveolar epithelial cell DNA repair after H2O2 exposure.” Am J Physiol 275, L780-787 (1998).
Takeoka M et al. “KGF facilitates repair of radiation-induced DNA damage in alveolar epithelial cells.” Am J Physiol 272, L1174-1180 (1997).
Narla S T et al. “Keratinocyte Growth Factor Reduces Injury and Leads to Early Recovery from Cyclophosphamide Bladder Injury.” Am J Pathol 190, 108-124, (2020).
Ulich T R et al. “Keratinocyte growth factor ameliorates cyclophosphamide-induced ulcerative hemorrhagic cystitis.” Cancer Res 57, 472-475 (1997).

### EXAMPLES

### Example 1. Synthesis of FGF7-p

FGF7-p (SEQ ID NO. 1) was chemically synthesized using methods known in the art for peptide synthesis. The FGF7-p peptide are purified and solubilized in 10% DMSO.

### Example 2. Treatment of Mice with FGF7-p

FVB/N mice were administered subcutaneous doses 5 mg/kg, 20 mg/kg, 40 mg/kg of FGF7-p, 5 mg/kg of full length FGF7, or 10% DMSO alone (vehicle). After 24 hours, 150 mg/kg of cyclophosphamide was administered to the mice. Mice were the sacrificed and bladder tissues examined for signs of urothelial injury at various times. At 48 hours, no signs of protection against urothelial apoptosis were detected in any of the FGF7-p-treated mice versus DMSO alone.

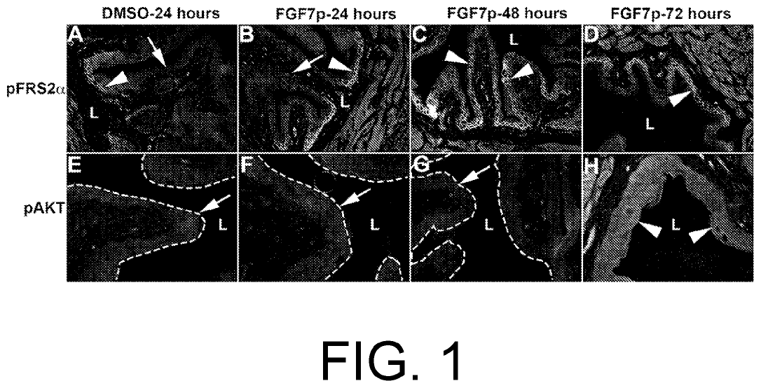

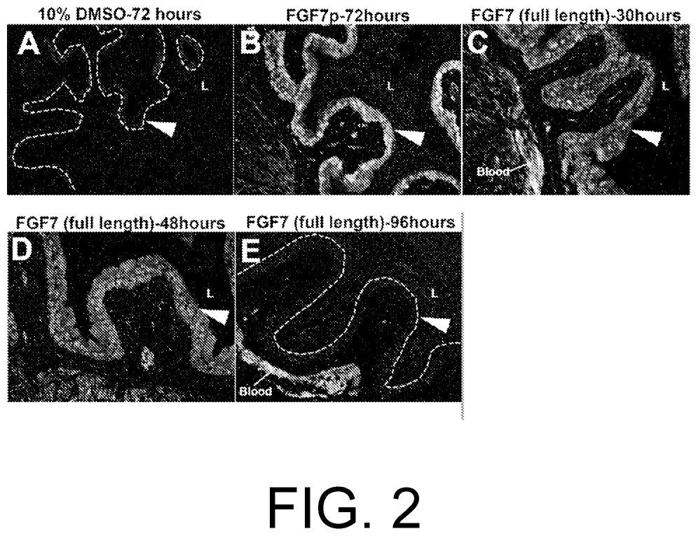

Uninjured mice were treated with 0.2 mL of 10% DMSO, 40 mg/kg FGF7-p, or 5 mg/ml full length FGF7 (n=2 mice per group, mice were about 25 g in weight) administered by subcutaneous injection. At the indicated times (Tables 1 and 2, FIGS. 1 and 2) bladders were harvested and immunostained for pFRS2α and pAKT, markers of urothelial protection. Immunostaining of bladder tissues from each group was repeated. Representative images are shown in FIGS. 1 and 2. Tables 1, 3, and 4 show the staining pattern for pFRS2α and pAKT over time in mice treated with full length FGF7. Tables 2, 3, and 4 show that in mice treating with FGF7-p, pFRS2α staining was similar to control at 24 hours, but robust at 48 hours and slightly diminished at 72 hours. Also as shown in Table 2, pAKT staining was absent at 24 and 48 hours, but robust at 72 hours after FGF7p. The data demonstrate show that, while onset of pFRS2α and pAKT is delayed in the FGF7-p group compared to full length FGF7, FGF7-p nevertheless stimulates robust expression of pFRS2α and pAKT in urothelial tissue.

After FGF7 binds to FGFR2IIIb, pFRSα appears first followed by pAKT. Activation of pAKT correlates with the anti-apoptotic/cytoprotective effects of FGF7 in the bladder urothelium.

Mice treated with DMSO control, full length FGF7, or FGF7-p were analyzed for activation of FGFR2IIIb and pAKT. Phosphorylated FRS2α and AKT levels were examined by immunostaining. After administration of full length FGF7, initial pFRS2α staining was observed as early as 15 hours later and pAKT staining was observed by 30 hours and peaked at 48 hours (see Tables 1-4 above). 48 hours after administration of 20 mg/kg or 40 mg/kg FGF7-p samples, strong FRS2α staining was observed. Less, but still significant pFRS2α staining was observed in mice treated with 5 mg/kg FGF7-p. The results indicate that onset of pFRS2α was delayed with FGF7-p in comparison with full length FGF7.

To assess whether pATK expression was also increase in FGF7-p treated mice, bladders were analyzed for pATK staining at 72 hours. To confirm pAKT staining was robust in bladder of FGF7-p of treated mice at 72 hours, additional staining for DMSO and FGF7-p (40 mg/kg) treated animals at 72 hours and staining on old samples from full length FGF7-treated samples (5 mg/kg) at 30 hours, 48 hours and 96 hours (n=2 mice per group) was performed. As seen in FIG. 2, pAKT staining in FGF7-p treated animals at 72 hours was as robust as full length FGF7 treated animals at 30 hours and 48 hours. pAKT staining in FGF7-p treated mice was high 72 hours (n=2 mice and staining repeated 3 times). Further, robust pAKT signal was seen throughout the urothelium. These results indicate activation of FGFR2IIIb and induction of pathways known to lead to protection of bladder urothelium against injury.

TABLE 1
pFRS2α and pAKT staining after giving full length FGF7

| stain | 15 h | 30 h | 48 h | 96 h |
|---|---|---|---|---|
| pFRS2α | ++ | +++ | + | − |
| pAKT | − | ++ | +++ | − |

TABLE 2
pFRS2α and pAKT staining after giving FGF7-p

| stain | 24 h | 48 h | 72 h |
|---|---|---|---|
| pFRS2α | Light/patchy (similar to control) | +++ | ++ |
| pAKT | — | − | +++ |

TABLE 3
pFRS2α staining after giving ligand

| Ligand | 15 h | 24 h | 30 h | 48 h | 72 h | 96 h |
|---|---|---|---|---|---|---|
| FGF7 full | ++ | n.d. | +++ | + | n.d. | − |
| length | | | | | | |
| FGF7-p | n.d. | Light/patchy | n.d. | +++ | ++ | n.d. |
| | | (similar to | | | | |
| | | control) | | | | |

n.d. = not done

TABLE 4
pAKT staining after giving ligand

| Ligand | 15 h | 24 h | 30 h | 48 h | 72 h | 96 h |
|---|---|---|---|---|---|---|
| FGF7 full | − | n.d. | ++ | +++ | n.d. | − |
| length | | | | | | |
| FGF7-p | n.d. | − | n.d. | − | +++ | n.d. |

n.d. = not done

### Example 3. FGF7-p Prevents Urothelial Apoptosis after Cyclophosphamide Treatment

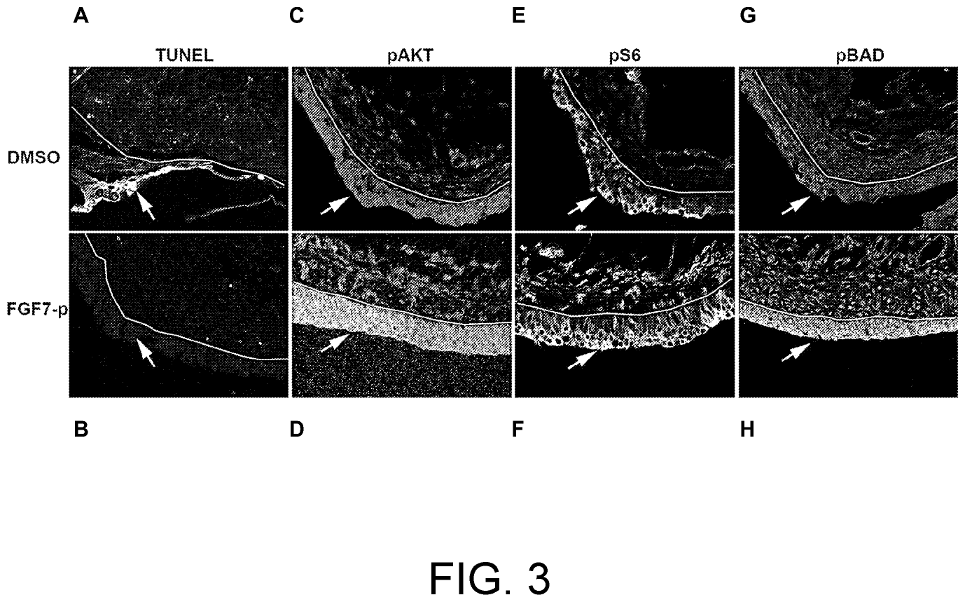

Mice were administered two doses of 20 mg/kg of subcutaneous (SQ) FGF7-p (or 10% DMSO vehicle) at 72 hours and 48 hours prior to intraperitoneal (IP) injection of 150 mg/kg cyclophosphamide (n=3 female mice per group). Mice were sacrificed and bladders were harvested 24 hours after cyclophosphamide injection. The bladders were fixed in 4% paraformaldehyde in phosphate buffered saline and embedded in paraffin. The paraffin embedded bladders were then serially sectioned with a microtome. Hematoxylin and eosin staining was performed on the sections. The staining showed significant urothelial injury with sloughing, hemorrhage, and inflammation in the DMSO control samples. However, there was almost no urothelial injury in the FGF7-p treated mice. Immunofluorescent terminal deoxynucleotidyl transferase dUTP nick end labeling (TUNEL) staining showed many sloughing apoptotic urothelial cells in the DMSO-treated mice. In contrast, FGF7-p-treated mice had intact, non-apoptotic cells (FIG. 3, A-B).

Immunostaining for phosphorylated (activated) AKT (pAKT) revealed weak urothelial staining in the DMSO-treated mice, but strong signal throughout the urothelium of FGF7-p-treated mice (FIG. 3, C-D).

Downstream targets of AKT, known to suppress apoptosis, have been found to be phosphorylated after infusion of full length FGF7 (not published). Immunostaining for phosphorylated p70S6 kinase (pS6), a readout of mTORC1 activation by AKT, showed weak staining in the DMSO-treated urothelium and much stronger staining in the FGF7-p-treated urothelium (FIG. 3, E-F).

Immunostaining for phosphorylated BAD (pBAD), a pro-apoptotic protein that is inactivated by AKT phosphorylation, revealed very weak staining in urothelium of DMSO-treated mice, but strong staining in urothelium of FGF7-p-treated mice (FIG. 3, G-H).

These findings show that the described FGF7-p blocks urothelial apoptosis and protects the urothelium from damage. FGF7-p treatment likely increases phosphorylation (activation) of AKT which in turn phosphorylates targets leading to mTORC1 activation and BAD suppression.

### Example 4. FGF7p Also Blocks Radiation-Induced Bladder Urothelial Cell Death

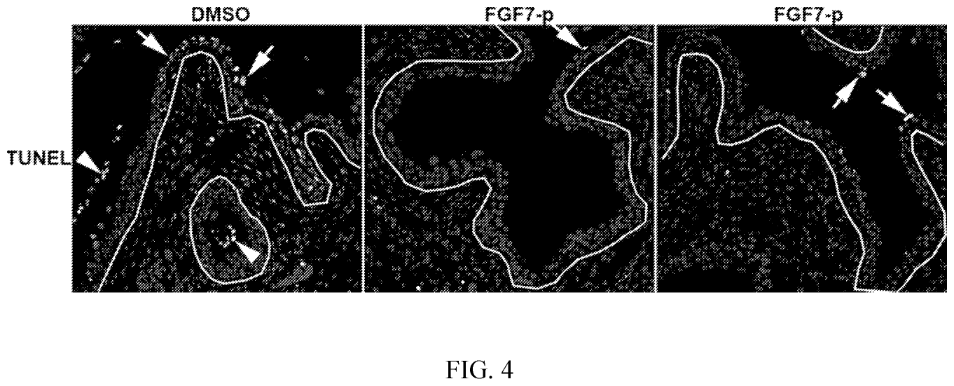

The ability of FGF7p to inhibit radiation injury as assesses. Two doses of FGF7p (20 mg/kg SQ) or 2.5% DMSO/PBS vehicle was administered to anesthetized female mice (N=2) at 72 hours and 48 hours prior to exposing the mice to 10 Gray (1000 rads) radiation. Instead of directly exposing bladders to irradiation, we transurethrally filled bladders with 300 μL of saline and focused the radiation beam over the bladder bulge on the depilated abdominal wall. One day later, we noted many TUNEL+ urothelial apoptotic cells in the vehicle-treated mice (FIG. 4, left panel). By comparison, FGF7p-treated mice had many regions with no or minimal urothelial apoptosis (FIG. 4, middle and right panels). Thus, similar to KGF, systemic FGF7p inhibited or blocked radiation-induced apoptosis.

1. An FGFR2IIIb agonist comprising an FGF7-p peptide, wherein the FGF7-p peptide is no more than about 130 amino acids in length and comprises an amino acid sequence differing by 0, 1, 2, or 3 amino acid substitutions, deletions, insertions, or combinations thereof from the amino acid sequence of SEQ ID NO. 1.

2. An FGFR2IIIb agonist comprising an FGF7-p peptide, wherein the FGF7-p peptide consists of a peptide differing by 0, 1, 2, or 3 amino acid substitutions, deletions, insertions, or combinations thereof from the amino acid sequence of SEQ ID NO. 1.

3. The FGFR2IIIb agonist of claim 1, wherein the FGF7-p peptide consists of a peptide differing by 3 amino acid substitutions, deletions, insertions, or combinations thereof from the amino acid sequence of SEQ ID NO. 1.

4. The FGFR2IIIb agonist of claim 1, wherein the FGF7-p peptide consists of a peptide differing by 2 amino acid substitutions, deletions, insertions, or combinations thereof from the amino acid sequence of SEQ ID NO. 1.

5. The FGFR2IIIb agonist of claim 1, wherein the FGF7-p peptide consists of a peptide differing by 1 amino acid substitution, deletion, or insertion from the amino acid sequence of SEQ ID NO. 1

6. The FGFR2IIIb agonist of claim 1, wherein the FGF7-p peptide consists of a peptide having the amino acid sequence of SEQ ID NO. 1.

7. The FGFR2IIIb agonist of any one of claims 1-5, wherein the 1, 2, or 3 amino acid substitutions are conservative amino acid substitutions.

8. A composition comprising the FGFR2IIIb agonist of any one of claims 1-7.

9. The composition of claim 8, further comprising one or more pharmaceutically acceptable excipients.

10. The composition of claim 9, wherein at least one of the one or more pharmaceutically acceptable excipients is dimethyl sulfoxide (DMSO).

11. The composition any one of claims 8-10, wherein the composition is an aqueous solution.

12. The composition of claim 11, wherein the aqueous solution is 0.1% to 10% DMSO.

13. The composition of claim 12, wherein the aqueous solution is 10% DMSO.

14. The composition of claim 8, wherein the composition is a lyophilized powder.

15. A nucleic acid encoding the FGF7-p peptide of any one of claims 1-7.

16. A method of treating a subject having epithelial damage or at risk of developing epithelial damage, comprising administering to the subject a pharmaceutically effective dose of the FGFR2IIIb agonist of any one of claims 1-7 or the composition of any one of claims 8-14.

17. The method of claim 16, wherein the subject has or is at risk of developing bladder urothelial damage, mucositis, gastrointestinal epithelial damage, lung epithelial damage, retinal epithelial damage, or skin epithelial damage.

18. The method of claim 15 or 16, wherein the subject has been administered or will be administered chemotherapy or radiation therapy or is undergoing autologous or allogeneic transplant.

19. The method of claim 18, wherein the subject has sarcoma, metastatic colorectal cancer, or head and neck cancer

20. The method of claim 18, wherein the chemotherapy comprises cyclophosphamide or ifosfamide chemotherapy.

21. The method of claim 16, wherein the subject has or is a risk of developing bladder pain syndrome.

22. The method of claim 16 or 17, wherein the subject has hematologic malignancies and/or is receiving myelotoxic therapy.

23. The method of any one of claims 16-22, wherein the subject has or is a risk of developing mucositis, severe mucositis, or severe oral mucositis.

24. The method of any one of claims 16-23, wherein the FGFR2IIIb agonist or the composition is administered by parenteral, local, direct injection, intraparenchymal, intramuscular, implantation, topical, systemic, intravascular, intravenous, intra-arterial, intraventricular, intralymphatic, transdermal, intracutaneous, intradermal, subdermal, subcutaneous, intraperitoneal, intravesical, intracranial, subdural, intrathecal, epidural, rectal, airway, nasal, oral, buccal, or sublingual administration.

25. The method of any one of claims 16-24, wherein the FGFR2IIIb agonist or the composition is administered prior to, concurrent with, and/or subsequent to onset or diagnosis of epithelial damage.

26. The method of any one of claims 16-22, wherein the FGFR2IIIb or the composition is administered prior to, concurrent with, and/or subsequent to administration to the patient of a therapy known to cause epithelial damage.

27. The method of any one of claims 16-22, wherein the FGFR2IIIb or the composition is administered prior to, concurrent with, and/or subsequent to onset or diagnosis of spinal cord injury, neurogenic bladder, non-neurogenic neurogenic bladder, or bladder pain syndrome.

28. A method of activating FGFR2IIIb in an epithelial cell or tissue, comprising contacting the epithelial cell or tissue with the FGFR2IIIb agonist of any one of claims 1-7 or the composition of any one of claims 8-14.

29. A method of increasing expression of phosphorylated fibroblast growth factor receptor substrate 2 (pFRS2α) in an epithelial cell or tissue comprising contacting the epithelial cell or tissue with the FGFR2IIIb agonist of any one of claims 1-7 or the composition of any one of claims 8-14.

30. A method of increasing expression of phosphorylated RAC-alpha serine/threonine-protein kinase (pAKT) in an epithelial cell or tissue comprising contacting the epithelial cell or tissue with the FGFR2IIIb agonist of any one of claims 1-7 or the composition of any one of claims 8-14.

31. A method of increasing growth and proliferation of epithelial cells or tissue comprising contacting the epithelial cell or tissue with the FGFR2IIIb agonist of any one of claims 1-7 or the composition of any one of claims 8-14.


SEQUENCE LISTING (2 sequences)

SEQ ID NO: 1 - protein, 19 aa, Homo sapiens
YASAKWTHNGGEMFVALNQ

SEQ ID NO: 2 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT